(19) United States
(12) Patent Application Publication
(10) Pub. No.: US 2021/0175426 A1
(43) Pub. Date: Jun. 10, 2021

(54) ORGANIC PHOTODETECTOR

(71) Applicant: SUMITOMO CHEMICAL CO., LTD, Tokyo (JP)

(72) Inventor: Nir Yaacobi-Gross, Cambridgeshire (GB)

(73) Assignee: Sumitomo Chemical Company Limited, Tokyo (JP)

(21) Appl. No.: 17/258,034

(22) PCT Filed: Jul. 2, 2019
(86) PCT No.: PCT/GB2019/051875

§ 371 Date: Jan. 5, 2021

(30) Foreign Application Priority Data
Jul. 6, 2018 (GB) 1811164.1

(51) Int. Cl. H01L 51/00

(52) U.S. Cl. CPC H01L 51/0036; H01L 51/0043; H01L 51/0047; H01L 51/4253; H01L 51/0074; H01L 51/0058; H01L 51/0068

(57) ABSTRACT

An organic photodetector comprising an anode; a cathode; and a photosensitive organic layer between the anode and cathode, wherein the photosensitive organic layer comprises an electron donor material and an electron acceptor compound of formula (I): R¹¹ and R¹² may be, e.g. C₁₋₂₀ alkyl and derivatives thereof or an aromatic or heteroaromatic group. R¹³-R¹⁶ may be, e.g. H, F, C₁₋₂₀ alkyl and derivatives thereof. R²⁰-R²³ independently in each occurrence is selected from the group consisting of H, C₁₋₂₀ alkyl and an electron-withdrawing group. Each X is independently O, S, Se or Te.

### FIELD

The disclosure relates to photoactive compounds and their use in organic electronic devices, in particular organic photodetectors.

### BACKGROUND

A range of organic electronic devices comprising organic semiconductor materials are known, including organic light-emitting devices, organic field effect transistors, organic photovoltaic (solar cell) devices and organic photodetectors (OPDs).

Cheng et al., “Next-generation organic photovoltaics based on non-fullerene acceptors”, Nat. Photonics, 2018, 12, 131-142 discloses non-fullerene acceptors for solar cells.

Yao et al., “Design and Synthesis of a Low Bandgap Small Molecule Acceptor for Efficient Polymer Solar Cells”, Adv. Mater., 2016, 28, 8283-8287 discloses non-fullerene acceptor IEICO for use in solar cells.

A drawback with OPDs is the presence of dark current, i.e. current flowing through the device in the absence of any photons incident on the device, which may affect the limit of detection of the device.

It is therefore an object of the invention to provide an OPD having low dark current.

It is a further object of the invention to provide an OPD having low dark current and good external quantum efficiency (EQE).

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

In many OPDs, dark current, which is the term used for current flowing through the OPD in the absence of any photons being incident on the OPD, may affect the limit of photon detection of the OPD.

The design considerations for an OPD differ from those for a solar cell (e.g. organic photovoltaics OPVs). Whilst high power conversion efficiency may be important for solar cells, one key focus in the design of photodetectors is reducing the dark current. By reducing the dark current, the specific detectivity may be increased. Preferably, the dark current is decreased without compromising the responsivity and/or EQE of the device. In addition, solar cells often require a relatively broad spectral response at wavelengths where most of the sun irradiance lays (e.g. λ<900 nm). Photodetectors, on the other hand, may operate in a specific or relatively narrow spectral band, which may be dependent upon a specific application. For example, the OPDs described herein may be suitable for the detection of light in the NIR regions of the electromagnetic spectrum, where irradiance from the sun is depleted (e.g. >900 or >940 nm).

The present inventors have found a class of acceptor compounds which may give low dark current as compared to the fullerene acceptor PCBM when used as an acceptor compound in an OPD.

The inventors have surprisingly found that the acceptor compounds provide EQEs of greater than 40% across a wide spectrum of wavelengths when used as acceptor compounds in OPDs.

The present inventors have further found that this class of acceptor compounds may be suitable for detection of light in the near infra-red region, in particular wavelengths of about 900-1000 nm. For example, OPDs comprising the acceptor compounds have been shown to exhibit an EQE that is greater than 40% at wavelengths of between about 900 nm and 950 nm. Sunlight is absorbed by atmospheric moisture at about 940 nm and so use of this class of acceptor compounds in a light source-OPD detector arrangement for detection of light having a wavelength in this range may be useable without shielding from sunlight. Because of absorption of sunlight in the atmosphere at these wavelengths, acceptor compounds according to the present disclosure may not be applicable to use in OPVs.

According to some embodiments there is provided an organic photodetector comprising an anode; a cathode; and a photosensitive organic layer between the anode and cathode, wherein the photosensitive organic layer comprises an electron donor material and an acceptor compound.

In some embodiments, the acceptor compound is a compound of formula (I):

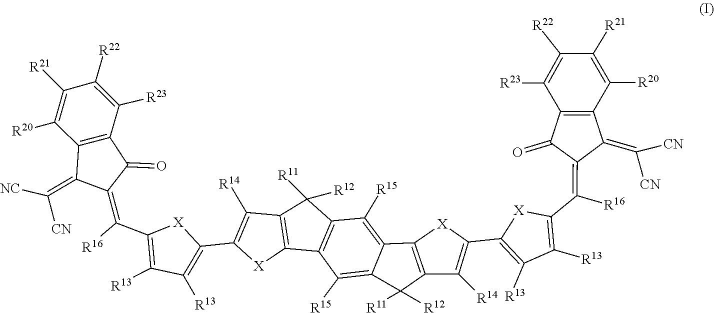

wherein:

each R¹¹ and R¹² is independently selected from the group consisting of:
C₁₋₂₀ alkyl wherein one or more non-adjacent, non-terminal C atoms may be replaced with O, S, CO or COO; one or more H atoms may be replaced with F; and a terminal C atom may be replaced with Ar² wherein Ar² is an unsubstituted or substituted aromatic or heteroaromatic group;
and
a group of formula (Ar¹)ₙ wherein Ar¹ in each occurrence is independently an aromatic or heteroaromatic group which is unsubstituted or substituted with one or more substituents and n is at least 1;

R¹³-R¹⁶ independently in each occurrence is selected from the group consisting of H, F and C₁₋₂₀ alkyl wherein one or more non-adjacent, non-terminal C atoms may be replaced with O, S, CO or COO and one or more H atoms may be replaced with F;

R²⁰-R²³ independently in each occurrence is selected from the group consisting of H, C₁₋₂₀ alkyl and an electron-withdrawing group; and each X is independently O, S, Se or Te.

By “non-terminal C atom” of an alkyl group as used herein is meant a carbon atom other than the methyl carbon atom of an n-alkyl group or the methyl carbon atoms of a branched alkyl group.

By “electron-withdrawing group” as used herein is meant a group having a positive para-substituent Hammett constant. Exemplary groups with a positive para-substituent Hammett constant are halogens selected from F, Cl, Br, I or CN, NO₂, CF₃ and C₁₋₁₂ fluoroalkyl.

The present inventors have found that use of compounds of formula (I) may give improved uniformity of a photosensitive layer formed by a solution deposition method, as compared to a photosensitive layer containing a fullerene acceptor.

In some embodiments there is provided a method of forming an organic photodetector comprising formation of an organic photosensitive layer over one of an anode and cathode and formation of the other of the anode and cathode over the organic photosensitive layer, wherein formation of the organic photosensitive layer comprises deposition of a formulation comprising an electron donor material and an electron acceptor compound of formula (I) dissolved or dispersed in one or more solvents and evaporation of the one or more solvents.

In some embodiments, there is provided a sensor comprising a light source and an organic photodetector, wherein the organic photodetector is configured to receive light from the light source.

In some embodiments, there is provided a method of detecting light comprising measurement of a photocurrent generated by light incident on an organic photodetector.

In some embodiments, there is provided a use of a compound of formula (I) in a photosensitive layer of an organic photodetector.

### DESCRIPTION OF DRAWINGS

The present disclosure is described in conjunction with the appended figures. It is emphasized that, in accordance with the standard practice in the industry, various features are not drawn to scale. In fact, the dimensions of the various features may be arbitrarily increased or reduced for clarity of discussion.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an organic photodetector according to some embodiments;

FIG. 2 is a graph of EQE vs wavelength for a device according to an embodiment and a comparative device; and

FIG. 3 is a graph of current density vs applied voltage in dark conditions for a device according to an embodiment and a comparative device.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

FIG. 1 illustrates an OPD according to some embodiments of the present disclosure. The OPD comprises a cathode 103 supported by a substrate 101, an anode 107 and a bulk heterojunction layer 105 disposed between the anode and the cathode comprising a mixture of an electron acceptor and an electron donor. Optionally, the bulk heterojunction layer consists of the electron acceptor and the electron donor. In the embodiment illustrated in FIG. 1, the OPD comprises a layer of material 106 which modifies the work function of the cathode 103. In other embodiments, this layer may or may not be present.

The OPD may comprise other layers not shown in FIG. 1. For example, the device may comprise a hole transport layer (HTL) located between the anode 107 and the heterojunction layer 105.

In other embodiments, the anode may be between the substrate and the bulk heterojunction layer and cathode.

In use, the photodetectors as described in this disclosure may be connected to a voltage source for applying a reverse bias to the device and a device configured to measure photocurrent.

In some embodiments, the photodetectors are part of a system comprising a plurality of photodetectors. For example, the photodetectors may be part of an array in an image sensor of a camera.

The voltage applied to the photodetector may be varied.

In some embodiments, the photodetector may be continuously biased when in use.

A sensor may comprise a light source and an OPD as described herein wherein the OPD is configured to receive light emitted from the light source. In some embodiments, the light from the light source may or may not be changed before reaching the light source. For example, the light may be filtered, down-converted or up-converted before it reaches the light source. Preferably, the sensor is configured to detect light having a wavelength in the range of about 900-1000 nm, optionally about 920-960 nm.

The inventors have also surprisingly found that when acceptor compounds described herein are used in OPDs, they may be suitable for detection of longer wavelength applications, in particular wavelengths of greater than 900 nm.

Preferably, the electron acceptor compounds described herein do not comprise a fullerene group, and are described hereinafter as “non-fullerene acceptors”.

An OPD comprising the acceptor compound may have an EQE of at least about 10%, 20%, 30%, 40%, 50%, 60%, 70%, 80% or 90% across a wide spectrum of wavelengths. Preferably, the EQE is greater than about 40% at wavelengths of between about 350 nm and 950 nm. The EQE may be measured as for the device described in Example 1.

The inventors have surprisingly found that incorporating acceptor compounds having a deep LUMO energy level relative to a fullerene derivative into an OPD can reduce dark current as compared to an OPD containing a fullerene compound as the acceptor.

In some embodiments, an OPD comprising the non-fullerene acceptor as described herein, produces a dark current that is at least 10 times less than fullerene derivative C₆₀PCBM.

In some embodiments, the acceptor compound is a compound of formula (I)

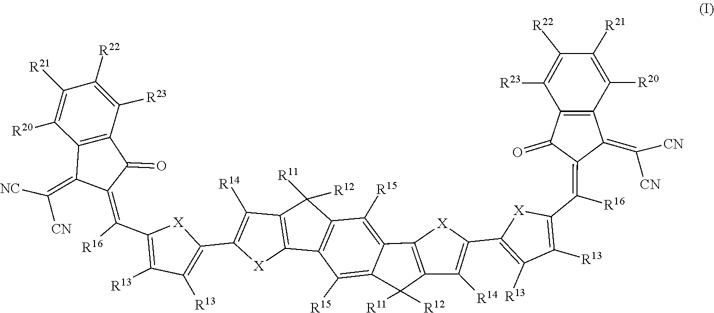

wherein:

each R¹¹ and R¹² is independently selected from the group consisting of:
C₁₋₂₀ alkyl wherein one or more non-adjacent, non-terminal C atoms may be replaced with O, S, CO or COO; one or more H atoms may be replaced with F; and a terminal C atom may be replaced with Ar² wherein Ar² is an unsubstituted or substituted aromatic or heteroaromatic group;
and
a group of formula (Ar¹)ₙ wherein Ar¹ in each occurrence is independently an aromatic or heteroaromatic group which is unsubstituted or substituted with one or more substituents and n is at least 1;

R¹³-R¹⁶ independently in each occurrence is selected from the group consisting of H, F and C₁₋₂₀ alkyl wherein one or more non-adjacent, non-terminal C atoms may be replaced with O, S, CO or COO and one or more H atoms may be replaced with F;

R²⁰-R²³ independently in each occurrence is selected from the group consisting of H, C₁₋₂₀ alkyl and an electron-withdrawing group; and each X is independently O, S, Se or Te.

Ar¹ may be a single ring or fused aromatic. Ar¹ may be selected from C₆₋₂₀ aromatic groups and 5-20 membered heteroaromatic groups. In some preferred embodiments, Ar¹ is phenyl or thiophene, more preferably phenyl.

n is preferably 1, 2 or 3, more preferably 1.

Ar² may be a single ring or fused aromatic. Ar¹ may be selected from C₆₋₂₀ aromatic groups and 5-20 membered heteroaromatic groups. In some preferred embodiments, Ar¹ is phenyl or thiophene, more preferably phenyl.

Each Ar¹ and Ar² is independently unsubstituted or substituted with one or more substituents. Substituents of Ar¹ may be selected from substituents R¹ wherein R¹ in each occurrence is independently selected from F, CN, NO₂ and C₁₋₂₀ alkyl wherein one or more non-adjacent, non-terminal C atoms may be replaced with O, S, CO or COO. C₁₋₂₀ alkyl is a preferred R¹ group.

Preferably, R¹¹ and R¹² are each a hydrocarbyl group.

R¹¹ and R¹² independently in each occurrence are preferably selected from C₁₋₂₀ alkyl, unsubstituted phenyl and phenyl substituted with one or more R¹ groups.

R¹³ in each occurrence is preferably selected from H, C₁₋₂₀ alkyl and C₁₋₁₆ alkoxyl. Optionally, one of two R¹³ groups bound to the same ring is H and the other R¹³ group is selected from C₁₋₂₀ alkyl and C₁₋₁₉ alkoxyl.

Preferably, at least one R¹⁴, more preferably each R¹⁴ is H.

Preferably, at least one R¹⁵, more preferably each R¹⁵ is H.

Preferably, at least one R¹⁶, more preferably each R¹⁶, is, H.

R²⁰-R²³ independently is preferably H or F. In preferred embodiments, at least one or at least 2 of R²⁰-R²³ is F.

Each X is preferably S.

Preferably, the compound of formula (I) has a LUMO of 3.85 eV or deeper as measured by square wave voltammetry. By “deeper” as used herein is meant further from vacuum level.

Preferably, the compound of formula (I) has a HOMO-LUMO bandgap of less than 1.5 eV.

Exemplary compounds of formula (I) are:

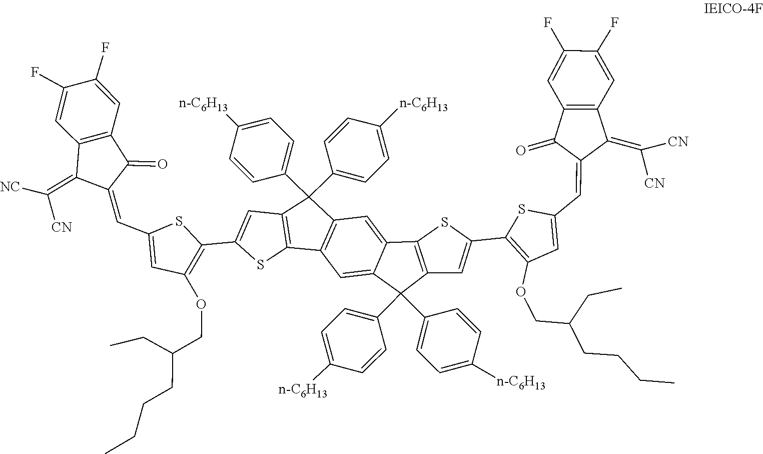

The donor compound (p-type) is not particularly limited and may be appropriately selected from electron donating materials that are known to the person skilled in the art, including organic polymers, oligomers and small molecules.

The donor compound can be a semiconducting polymer.

In a preferred embodiment the p-type donor compound comprises an organic conjugated polymer, which can be a homopolymer or copolymer including alternating, random or block copolymers. Preferred are non-crystalline or semi-crystalline conjugated organic polymers. Further preferably the p-type organic semiconductor is a conjugated organic polymer with a low bandgap, typically between 2.5 eV and 1.5 eV, preferably between 2.3 eV and 1.8 eV. As exemplary p-type donor polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bis selenophene), poly(terselenophene), polythieno[2,3-b]thiophene, polythieno[3,2-b]thiophene, polybenzothiophene, polybenzo[1,2-b:4,5-b′jdithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned. Preferred examples of p-type donors are copolymers of polyfluorenes and polythiophenes, each of which may be substituted, and polymers comprising benzothiadiazole-based and thiophene-based repeating units, each of which may be substituted. It is understood that the p-type donor may also consist of a mixture of a plurality of electron donating materials.

In some embodiments, the weight of the donor compound to the acceptor compound is from about 1:0.5 to about 1:2.

Preferably, the weight ratio of the donor compound to the acceptor compound is about 1:1 or about 1:1.5.

At least one of the first and second electrodes is transparent so that light incident on the device may reach the bulk heterojunction layer. In some embodiments, both of the first and second electrodes are transparent.

Each transparent electrode preferably has a transmittance of at least 70%, optionally at least 80%, to wavelengths in the range of 300-900 nm.

In some embodiments, one electrode is transparent and the other electrode is reflective.

Optionally, the transparent electrode comprises or consists of a layer of transparent conducting oxide, preferably indium tin oxide or indium zinc oxide. In preferred embodiments, the electrode may comprise poly 3,4-ethylenedioxythiophene (PEDOT). In other preferred embodiments, the electrode may comprise a mixture of PEDOT and polystyrene sulfonate (PSS). The electrode may consist of a layer of PEDOT:PSS.

Optionally, the reflective electrode may comprise a layer of a reflective metal. The layer of reflective material may be aluminium or silver or gold. In some embodiments, a bi-layer electrode may be used. For example, the electrode may be an indium tin oxide (ITO)/silver bi-layer, an ITO/aluminium bi-layer or an ITO/gold bi-layer.

The device may be formed by forming the bulk heterojunction layer over one of the anode and cathode supported by a substrate and depositing the other of the anode or cathode over the bulk heterojunction layer.

The area of the OPD may be less than about 3 cm², less than about 2 cm², less than about 1 cm², less than about 0.75 cm², less than about 0.5 cm² or less than about 0.25 cm². The substrate may be, without limitation, a glass or plastic substrate. The substrate can be described as an inorganic semiconductor. In some embodiments, the substrate may be silicon. For example, the substrate can be a wafer of silicon. The substrate is transparent if, in use, incident light is to be transmitted through the substrate and the electrode supported by the substrate.

The substrate supporting one of the anode and cathode may or may not be transparent if, in use, incident light is to be transmitted through the other of the anode and cathode.

The bulk heterojunction layer may be formed by any process including, without limitation, thermal evaporation and solution deposition methods.

Preferably, the bulk heterojunction layer is formed by depositing a formulation comprising the acceptor material and the electron donor material dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

The organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected. The sensor may be, without limitation, a gas sensor, a biosensor, an X-ray imaging device, a motion sensor (for example for use in security applications) a proximity sensor or a fingerprint sensor. The photodetector may be form part of a 1D or 2D array in an image sensor. For example, the photodetector may be part of an array of photodetectors in a camera image sensor.

### EXAMPLES

### Materials

Devices were formed using Donor Polymer 1 and either fullerene C60PCBM or IEICO-4F as the acceptor.

Donor Polymer 1 has the structure:

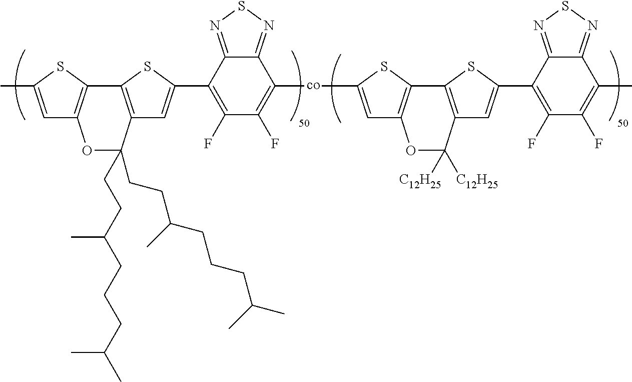

### LUMO Measurement

The LUMO energy levels reported herein were determined using square wave voltammetry (SWV) at room temperature in solution. In square wave voltammetry, the current at a working electrode is measured while the potential between the working electrode and a reference electrode is swept linearly in time. The difference current between a forward and reverse pulse is plotted as a function of potential to yield a voltammogram. The apparatus to measure HOMO or LUMO energy levels by SWV may comprise a cell containing tertiary butyl ammonium perchlorate or tertiary butyl ammonium hexafluorophosphate in acetonitrile; a glassy carbon working electrode; a platinum counter electrode and a leak free Ag/AgCl reference electrode. Ferrocene is added directly to the existing cell at the end of the experiment for calculation purposes where the potentials are determined for the oxidation and reduction of ferrocene versus Ag/AgCl using cyclic voltammetry (CV).

LUMOs of the acceptor compounds were measured by square wave voltammetry using a CHI 660D Potentiostat with a 3 mm diameter glassy carbon working electrode Leak free Ag/AgCl reference electrode Pt wire auxiliary or counter electrode and 0.1 M tetrabutylammonium hexafluorophosphate in acetonitrile.

The sample was dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

The square wave voltammetry experiment may be run at 15 Hz frequency; 25 mV amplitude and 0.004V increment steps. Results are calculated from 3 freshly spun film samples for both the HOMO and LUMO data.

All experiments were run under an argon gas purge.

C₆₀PCBM has a LUMO level of 3.81 eV

IEICO-4F has a LUMO level of 3.91 eV.

Comparative Device 1

A device having the following structure was prepared:

Cathode/Donor:Acceptor layer/Anode

A glass substrate coated with a patterned layer of indium-tin oxide (ITO) was treated with polyethyleneimine (PEIE) to modify the work function of the ITO.

A ca. 800 nm thick bulk heterojunction layer of a mixture of Donor Polymer 1 and acceptor compound C₆₀PCBM was deposited over the modified ITO layer by bar coating from a 1,2,4-trimethylbenzene: 1,2-dimethoxybenzene solvent mixture in a donor:acceptor mass ratio of 1:2.

An anode (Clevios HIL-E100) available from Heraeus was formed over the donor/acceptor mixture layer by spin-coating.

### Example 1

A device was formed as described for Comparative Device 1 except that a bulk heterojunction layer of a mixture of Donor Polymer 1 and IEICO-4F as the acceptor compound was deposited over the modified ITO layer by spin-coating from 1,2,4-trimethylbenzene:benzyl benzoate in a donor:acceptor weight ratio of 1:1.5.

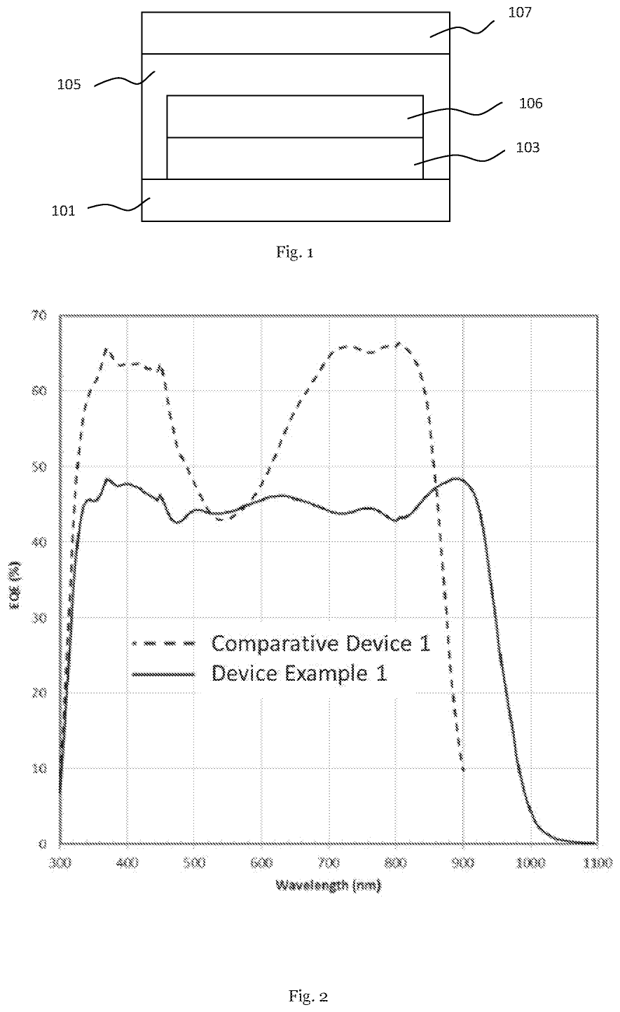

External quantum efficiencies (EQE) of the devices were measured in reverse bias (2V). With reference to FIG. 2, absorption for Device Example 1 extends further into the near-infrared region than Comparative Device 1.

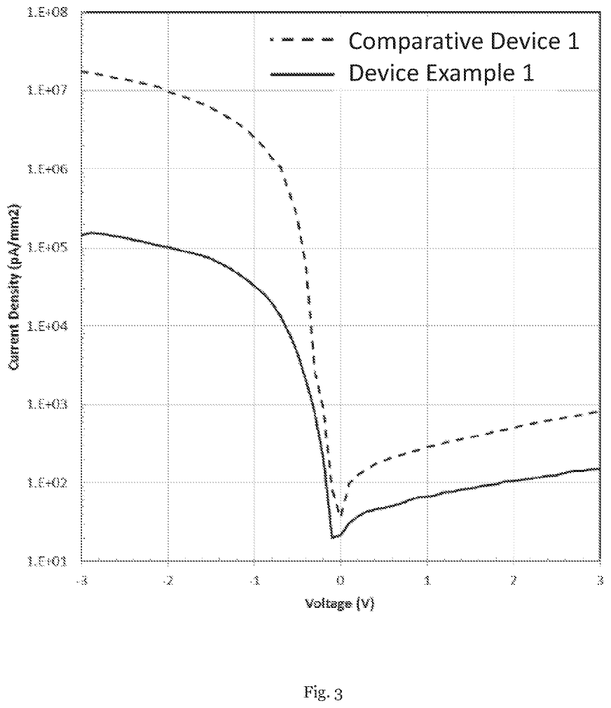

With reference to FIG. 3, dark current of Device Example 1 is significantly lower than that of Comparative Device 1, despite the LUMO of the acceptor of Device Example 1 being lower than that of Comparative Device 1.

1. An organic photodetector comprising an anode; a cathode; and a photosensitive organic layer between the anode and cathode, wherein the photosensitive organic layer comprises an electron donor material and an electron acceptor compound of formula (I): 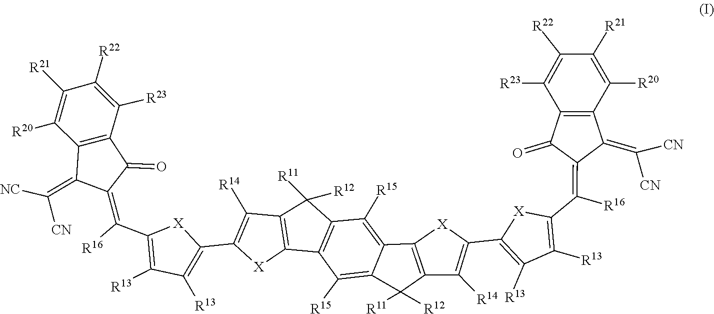
wherein:
each R¹¹ and R¹² is independently selected from the group consisting of:
C₁₋₂₀ alkyl wherein one or more non-adjacent, non-terminal C atoms may be replaced with O, S, CO or COO; one or more H atoms may be replaced with F; and a terminal C atom may be replaced with Ar² wherein Ar² is an unsubstituted or substituted aromatic or heteroaromatic group;
and
a group of formula (Ar¹)ₙ wherein Ar¹ in each occurrence is independently an aromatic or heteroaromatic group which is unsubstituted or substituted with one or more substituents and n is at least 1;
R¹³, R¹⁴ and R¹⁶ independently in each occurrence is selected from the group consisting of H, F and C₁₋₂₀ alkyl wherein one or more non-adjacent, non-terminal C atoms may be replaced with O, S, CO or COO and one or more H atoms may be replaced with F;
R¹⁵ in each occurrence is H;
R²⁰-R²³ independently in each occurrence is selected from the group consisting of H, C₁₋₂₀ alkyl and an electron-withdrawing group; and
each X is independently O, S, Se or Te,
and wherein the organic photodetector is connected to a voltage source configured to apply a reverse bias to the organic photodetector.

2. An organic photodetector according to claim 1 wherein at least one R¹¹ group and/or at least one R¹² group is a group of formula (Ar¹)ₙ.

3. An organic photodetector according to claim 2 wherein (Ar¹)ₙ is phenyl which is unsubstituted or substituted with one or more substituents.

4. An organic photodetector according to claim 1 wherein R¹³ in each occurrence is independently selected from H, C₁₋₂₀ alkyl and C₁₋₁₉ alkoxyl.

5. An organic photodetector according to claim 1 wherein each R¹⁴ is H.

6. (canceled)

7. An organic photodetector according to claim 1 wherein each R¹⁶ is H.

8. An organic photodetector according to claim 1 wherein each R²⁰-R²³ independently is H or F.

9. An organic photodetector according to claim 8 wherein each R²⁰, each R²¹, each R²² and/or each R²³ is F.

10. An organic photodetector as claimed in claim 1, wherein the weight ratio of the donor compound to the acceptor compound is from about 1:0.5 to about 1:1.2.

11. A method of forming an organic photodetector according to claim 1 comprising formation of the organic photosensitive layer over one of the anode and cathode and formation of the other of the anode and cathode over the organic photosensitive layer, wherein formation of the organic photosensitive layer comprises deposition of a formulation comprising the electron donor material and the electron acceptor compound dissolved or dispersed in one or more solvents and evaporation of the one or more solvents.

12. A sensor comprising a light source and an organic photodetector as claimed in claim 1, wherein the organic photodetector is configured to receive light emitted from the light source.

13. A sensor according to claim 12 wherein the organic photodetector is configured to receive light having a wavelength of 900-1000 nm.

14. A method of detecting light comprising measurement of a photocurrent generated by light incident on an organic photodetector as claimed in claim 1.

15. A method of detecting light as claimed in claim 14, wherein the method comprises measurement of the photocurrent generated by light incident on the organic photodetector and emitted from a light source of a sensor comprising the light source and the organic photodetector.

16. Use of a compound of formula (I) as defined in claim 1 as an acceptor in a photosensitive layer of an organic photodetector comprising the acceptor and a donor material.

17. A use as claimed in claim 16 for reducing dark current.
